(19) United States
(12) Patent Application Publication
(10) Pub. No.: US 2018/0064582 A1
(43) Pub. Date: Mar. 8, 2018

(54) DIFFERENTIATED LYMPH DRAINAGE GARMENT

(71) Applicants: Ruggiero DEFENTE, Barletta (BT) (IT); Salvatore PAPARELLA, Barletta (BT) (IT)

(72) Inventors: Ruggiero DEFENTE, Barletta (BT) (IT); Salvatore PAPARELLA, Barletta (BT) (IT)

(21) Appl. No.: 15/558,056

(22) PCT Filed: Mar. 17, 2016
(86) PCT No.: PCT/IB2016/000311

§ 371 Date: Sep. 13, 2017

(30) Foreign Application Priority Data
Mar. 20, 2015 (IT) BA2015A000025

(51) Int. Cl. A61F 13/08; A41B 11/00

(52) U.S. Cl. CPC A61F 13/08; A41B 11/14; A61H 2205/10; A41B 11/003

(57) ABSTRACT

In the field of medical devices for the treatment of diseases related to blood circulation, a garment is provided which is capable of improving the lymph drainage of adipose tissues of the legs. The differentiated lymph drainage garment is characterized by at least two areas, a front one and a back one, contiguous to each other, each made with a fabric divided into bands having a decreasing elasticity from the bottom upwards. The elasticity of the bands in the front area is, at the same height, different from, or only partially coincident with the elasticity of the bands of the back area.

The invention relates to the field of medical devices for the treatment of diseases related to blood circulation and, more specifically, the object of the present invention are pants capable of improving the lymph drainage of adipose tissues of the legs.

As is known, the term lymphedema defines an anomaly of the lymphatic system which causes an abnormal accumulation of lymph especially in the limbs.

The triggering factors of this condition of accumulation of lymph can be, in addition to lifestyle, too tight clothing which excessively compress the limbs, obstructing both the blood and the lymphatic circulation, giving rise to the so-called orange peel skin and imperfections such as fat deposits on thighs, hips and buttocks.

A poor hemolymph circulation causes a poor blood and lymph inflow to the tissues, resulting in poor nutrition of the same, poor oxygen supply and reduced ability to eliminate toxins, resulting in the accumulation of harmful substances.

Techniques for improving or at least maintaining the health of the blood and lymphatic circulation are known. Aside from the surgical techniques used for advanced clinical cases, as well as the pharmacological methodologies, the currently most used techniques are those related to lymphatic drainage massage and to the elastic compression technology; in particular, the latter involve the use of elastic stockings or bands.

Bands, definitely unsightly and unpleasant to patients, are very useful and are used for more serious diseases, thus becoming irreplaceable.

However, their use is mostly temporary, to allow healing of an injury (immediate post-operative, after sclerotherapy, superficial thrombophlebitis), or to return the system back to balance (edema, post-thrombotic syndrome, ulcers).

They are also used to eliminate the swelling (edema) present in the legs as a result of venous hypertension.

They are also useful in the treatment of ulcers, especially in the early stage.

However, said bands have a number of drawbacks and, in particular, in addition to the blemishes listed above, they are difficult to apply by the nurses and by the patient or his/her relatives.

In contrast, stockings are better appreciated by doctors and patients as they are aesthetically nicer and apparently easier to wear. However, they have the drawback of being less effective than the bands and have a higher cost. Moreover, heavier stockings are difficult to wear, especially for older people.

Pants are also known which are made with a fabric divided into bands with different elasticity, decreasing from the bottom upwards.

The object of the present invention is to provide a device which provides a lymphatic drainage massage to improve the blood and lymphatic circulation by means of pants which provide a decreasing graduated compression starting from the ankles.

A further object is to provide a garment which solves the problems related to the inherent blemishes of known devices and which is particularly suitable for improving the blood and lymphatic circulation, helping to improve aesthetic imperfections such as cellulite and the so-called orange peel skin.

Yet another object is to provide a piece of clothing with elasticity decreasing from the top downwards but differentiated between the front and back part. The difference between the two panels allows preserving the graduated compression, respecting the anatomical front-back differences, enhancing the fit and the modeling effect and comfort, as well as creating in the band compression, thanks to the difference between the front and back bands, a further intermediate compression band (since it is the average of the two bands) which softens the pressure change between one band and the other.

Yet another object is to provide pants with elastic bands the fabric of which comprises mineral components in order to obtain a garment capable of producing heat when wet by perspiration or by the physiological hydrolipid film, and retain it and release it slowly.

Another object is to provide a garment the fabric of which comprises mineral components capable of releasing infrared rays, with positive effects on the vascular health in general.

The differentiated lymph drainage garment according to the present invention is described hereinafter, in a preferred non-limiting embodiment of further developments in the scope of the invention itself, with the aid of the accompanying drawing which illustrates the following figure:

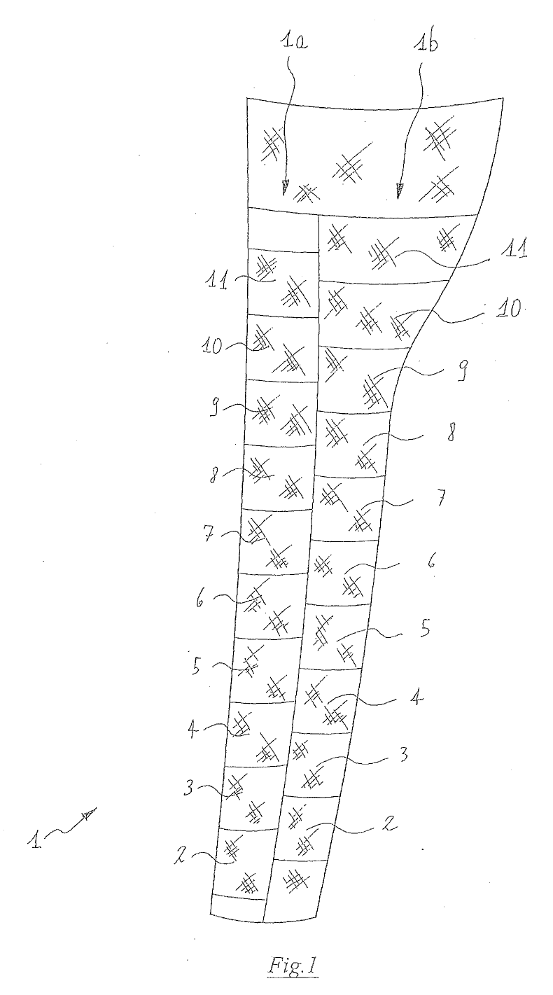

FIG. 1, a side view of a leg of pants made according to the present invention, wherein the bands with upwards decreasing concentration of the elastomers are highlighted.

According to the present invention, garment 1 uses a fabric formed by areas whose concentration of elastomers gradually varies.

More in particular, as shown in the accompanying figure, the garment which for example is pants, comprises a front part 1a and a back part 1b, each comprising a plurality of bands from 2 to 11 whose elasticity, i.e. the degree of compression capable of exerting on the user's body, does not coincide but, in both cases, decreases from the bottom upwards.

The progressive elasticity is achieved by inserting a greater number of elastomers in the weft. The maximum area of pressure, about 20 mmhg, is made at the front of the ankle, i.e. in the fabric band which has the highest concentration of elastomers and it progressively decreases in the bands close to the crotch of the pants, for example at knee height, the pressure exerted by band 6 is about 10 mmHg, in contrast band 11 in the proximity of the waist will exert a pressure of about 1 mmhg.

The slow graduated, decreasing and constant pressure only partially coinciding at the front with respect to the back, supports and facilitates the lymphatic circulation, ensuring a stimulus of the same, preventing the lymphatic stasis which leads to the problems already mentioned. The lymph drainage pants 1, through the walking of its user, create a pumping with an effect on the lymphatic vessels which contract and move up the lymph, draining the connective tissue. The beneficial effects of the garment are also amplified by the fact that the fabric comprises zeolite and steatite components.

According to the present invention, garment 1 is made according to the following steps:

1. Tailoring a garment comprising a front fabric piece 1a, formed by a plurality of bands with different elasticity, and a back fabric piece 1b, which is also formed by a plurality of bands with different elasticity; the bands of the front fabric piece 1a and the bands of the back fabric piece 1b, with the same elasticity, having at least one contact point;

2. Spraying a mixture consisting of resin and zeolite on the inside and/or outside of the garment;

3. Subsequently spraying a mixture consisting of resin and steatite on the inside and/or outside of the same garment;

4. Fixing the compound of resin and color through a wash at the dry cleaner's. In order to facilitate the lymphatic and venous circulation using the peculiarities of blood and lymph, which tend to move from the cold areas of the body to those warmer, the spraying of minerals is not uniform but more concentrated in certain areas of the garment.

1. A differentiated lymph drainage garment characterized by at least two areas, a front one and a back one contiguous to each other, each made with a fabric divided into bands having a decreasing elasticity from the bottom upwards; the elasticity of the bands in the front area being, at the same height, different from or only partially coincident with the elasticity of the bands of the back area.

2. A differentiated lymph drainage garment according to claim 1, wherein the fabric of the at least one front area comprises at least three bands with different elasticity, each of which is contiguous to one of the bands that make up the fabric forming the back area, having the same elasticity degree.

3. A differentiated lymph drainage garment according to claim 1, wherein the fabric used for making the garment comprises a variable percentage of zeolite and steatite mineral.

4. A method for making a differentiated lymph drainage garment according to claim 1, characterized by the following steps:
tailoring the garment comprising a front fabric piece, formed by a plurality of bands with different elasticity, and a back fabric piece, which is also formed by a plurality of bands with different elasticity; the bands of the front fabric piece and the bands of the back fabric piece, with the same elasticity, having at least one contact point;
spraying a mixture comprising resin and zeolite on the inside and/or outside of the garment;
subsequently spraying a mixture comprising resin and steatite on the inside and/or outside of the same garment; and
fixing the compound of resin and color through a wash at a dry cleaner's.
